(19) United States
(12) Patent Application Publication
(10) Pub. No.: US 2007/0173697 A1
(43) Pub. Date: Jul. 26, 2007

(54) COMBINED FLEXIBLE AND RIGID INTUBATING VIDEO LARYNGOSCOPE

(75) Inventors: Paul Dutcher, Pittsford, NY (US); Farhan Taghizadeh, Albuquerque, NM (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Rochester Medical Center, Rochester, NY (US)

(21) Appl. No.: 11/465,073

(22) Filed: Aug. 16, 2006

Related U.S. Application Data
(60) Provisional application No. 60/709,189, filed on Aug. 17, 2005.

(51) Int. Cl. A61B 1/267

(52) U.S. Cl. 600/188

(57) ABSTRACT

A combined flexible and rigid intubating video laryngoscope. The laryngoscope has a handle, and a rigid blade, having a proximal end connectible to the handle and a distal end projecting therefrom. The laryngoscope also has a flexible video endoscope having a distal end connectible near the distal end of the blade, the distal end of the endoscope being movable relative to the distal end of the blade, so as to enable the endoscope to obtain a variable view including a more anterior anatomical view of a patient's vocal cords during direct laryngoscopy.

### CROSS-REFERENCES TO RELATED APPLICATIONS

This application claims priority to U.S. Provisional Patent Application No. 60/709,189, filed Aug. 17, 2005, the disclosure of which is hereby incorporated by reference in its entirety for all purposes.

### BACKGROUND OF THE INVENTION

The present invention relates generally to a laryngoscope, and more particularly to a combined flexible and rigid intubating laryngoscope.

A laryngoscope is a medical instrument for examining the larynx. The larynx is a cartilaginous structure at the top of the trachea that contains elastic vocal cords that are the source of the vocal tone in speech. The trachea is a membranous tube with cartilaginous rings that conveys inhaled air from the larynx to the bronchi. A laryngoscope is used to obtain a view of the glottis (i.e., the vocal apparatus of the larynx, consisting of the true vocal cords and the opening between them) by direct laryngoscopy. A standard adult laryngoscope consists of a handle and a blade with a light source.

Many varieties of laryngoscopes are commercially available. These include the rigid blade-type laryngoscopes as well as video endoscopes. One improvement in laryngoscopes has been directed towards the use of image capture devices and their integration with laryngoscopes. As such, video-enhanced laryngoscopes are now available as tools that allow the individual the advantage of looking up, and guiding in the endotracheal tube while looking at a monitor. These improved laryngoscopes use image capture devices at the end of conventionally designed laryngoscopes, and project the captured images on video screens.

The enhanced rigid fiberoptic endoscopes and video laryngoscopes are exemplary prior art devices for intubating patients and establishing an airway that integrate fiberoptic and video technology into traditional laryngoscopes. Intubation refers to the placement of a tube into an external or internal orifice of the body. Although the term can refer to endoscopic procedures, it is most often used to denote tracheal intubation. Tracheal intubation can be performed by direct laryngoscopy (conventional technique), in which a laryngoscope is used to obtain a view of the glottis. A tube is then inserted under direct observation. This technique can only be employed if the patient is comatose (unconscious) or under general anesthesia.

However, use of such fiberoptic and/or video laryngoscopes still does not address the chronic problems that are associated with not being able to visualize a desired part of the larynx, since these devices still don't allow for a good visualization of the vocal chords. While anatomical variation, often assigned various classifications, determines the potential for a “best view” of the vocal chords, this perspective is not always optimal. Physician skill variation can determine the extent of a “best view,” but in many patients with suboptimal anatomy this view has serious limitations.

Prior art enhanced laryngoscopes may have improved how a physician views the larynx, yet these devices do not give physicians the ability to change their view of the larynx at the point of intubation. For example, while fiberoptic technologies exist to allow for changes in the view, such techniques are cumbersome and often are used in only the worst patients. One can not freely intubate patients with the non-scope hand using fiberoptic technologies, making it impractical for common use.

With regard to rigid scopes, there is very little variation in the “best view” a physician is able to obtain. Based in part on experience in otolaryngology where physicians are often called for the worst airways, the single “best view” of these scopes often leads to lost airways and patient complications. This view invariably is too far posterior, not allowing for good visualization of the vocal chords, and thus subsequently complicating intubation.

There is therefore a need for a laryngoscope that is both easy to use to intubate a patient that also provides for an adjustable view of the patient's vocal cords at the point of intubation.

### BRIEF SUMMARY OF THE INVENTION

The present invention provides a combined flexible and rigid intubating video laryngoscope. The laryngoscope has a handle, and a rigid blade, having a proximal end connectible to the handle and a distal end projecting therefrom. The laryngoscope also has a flexible video endoscope having a distal end connectible near the distal end of the blade, the distal end of the endoscope being movable relative to the distal end of the blade, so as to enable the endoscope to obtain a variable view including a more anterior anatomical view of a patient's vocal cords during direct laryngoscopy.

In one aspect, the laryngoscope also includes a dial operatively connected with the flexible video endoscope, where the dial is configured to move the distal end of the endoscope relative to the distal end of the blade.

In another aspect, the flexible video endoscope has optic fibers for image transmission and light fibers for airway illumination.

In another aspect, the flexible video endoscope incorporates an imaging array. The imaging array may be a charge-coupled device imaging array.

In another aspect, the laryngoscope also includes means for flowing a gas to the distal end of the flexible video endoscope. The means for flowing a gas can include a flow channel integrated with the video endoscope.

In another aspect, the laryngoscope also includes suction means located near the distal end of the blade, so as to allow for secretion clearance during intubation.

In another aspect, the laryngoscope also includes display means operatively connectible with the video endoscope for displaying the visual field observable by the distal end of the video endoscope. The display means can be a display screen detachably connected with the handle. The display screen can also be positionable to allow the intubator simultaneous viewing of the visual field directly and the visual field indirectly through the display screen. The laryngoscope can also include power supply means placed inside the handle for powering the display screen.

In another aspect, the laryngoscope also includes illumination means placed inside the handle for illuminating the visual field observable by the distal end of the video endoscope. The illumination means can be a halogen light source operatively connectible with the proximal end of the video endoscope. And the power supply means placed inside the handle can be used for powering the illumination means.

In another aspect, the laryngoscope also includes power supply means placed inside the handle for powering the video endoscope.

These and other embodiments of the present invention, as well as its advantages and features, are described in more detail in conjunction with the description below and the attached figures.

### BRIEF DESCRIPTION OF THE DRAWINGS

FIG. 1 is a simplified exemplary diagram of the combined flexible and rigid intubating video laryngoscope in accordance with one embodiment of the present invention.

FIG. 2 is a more detailed exemplary diagram for the combined flexible and rigid intubating video laryngoscope of FIG. 1.

FIG. 3 shows the laryngoscope view using prior art video laryngoscope designs on the left, where the camera is on the tip and looks posteriorly, and the laryngoscope view using the combined flexible and rigid intubating video laryngoscope of FIG. 1, which looks anteriorly.

### DETAILED DESCRIPTION OF THE INVENTION

The embodiments of the present invention provide a combined flexible and rigid intubating laryngoscope that includes several novel features. A first novel feature is directed towards the use of a small display screen, such as a liquid quartz display (LCD) screen that is removably attached to the scope. The display screen is similar to those found on commercially available video cameras. With the small display screen, the line of sight can be upward, without excess wires leading to a non-attached video screen, such as those used with video or other video-based laryngoscopes or endoscopes. This screen can be detached to allow for the scope to be cleaned between intubations. The screen is operatively connected with an imaging device (e.g., a CCD camera) at the end of the fiberoptic scope.

A second novel feature is related to the laryngoscope's a rigid and curved blade. Integrated into the tip of the blade section of the laryngoscope in accordance with the embodiments of the present invention is a flexible endoscope having a toggle or a dial to enable the endoscope's tip to be movable and/or rotatable and thus provide a more anterior anatomical view of the larynx. This flexible endoscope is operatively coupled with an imaging device (e.g., a CCD camera).

A third novel feature is directed towards having a power source (e.g., rechargeable battery) and an illumination source located in or embedded into the laryngoscope handle, to light the flexible scope.

A fourth novel feature is directed towards having a suction capability at the laryngoscope tip, to allow for secretion clearance at the time of intubation. In addition to a suction flow path, a flow path for flowing a gas to the distal end of the flexible video endoscope can also be provided. The flow path for flowing a gas can be a flow channel integrated with the video endoscope.

The combined flexible and rigid intubating laryngoscope in accordance with the embodiments of the present invention provides physicians with the ability to toggle and/or modify their view and get the patient intubated in a timely fashion. It gives physicians the comfort of a video display screen disposed on the laryngoscope, having local and rechargeable lighting.

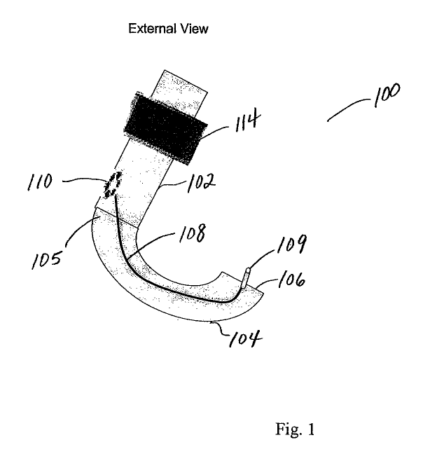

FIG. 1 shows a simplified exemplary diagram of the combined flexible and rigid intubating video laryngoscope 100 in accordance with one embodiment of the present invention. The laryngoscope includes a handle 102 and a rigid curved blade 104. The blade 104 is connectible with the handle 102 at the blade's proximal end 105. The distal end of the blade 106 is free. A flexible video endoscope 108 has a distal end 109 ending near the distal end of the blade 106. The flexible video endoscope 108 can be a fiberoptic scope having a CCD imaging array. The distal end of the endoscope 109 is movable relative to the distal end of the blade 106. The distal end of the endoscope 109 can move up/down and sideways relative to the distal end of the blade 106. At the proximal end of the endoscope a dial 110, or equivalent mechanism is located on the handle 102. The dial 110 is used to move the distal tip 109 of the video endoscope 108 relative to the distal end of the blade 106, so as to enable the endoscope to obtain a variable view including a more anterior anatomical view of a patient's vocal cords during direct laryngoscopy. Furthermore, the distal tip 109 is also movable to enable the manipulation of the airway. In addition, a display screen 114 is detachably and removably connected with the handle 102. The removable screen allows the laryngoscope to be easily cleaned. The screen can be re-attached for a subsequent use.

The screen 114 is used to display the visual filed observable by the distal end of the video endoscope 109. The display screen's orientation with respect to handle 102 is adjustable, in a manner similar to electronic still or video cameras' display screens. In addition, the laryngoscope 100 can also include a flow channel for flowing a gas to the distal end of the flexible video endoscope. The flow channel for flowing a gas can be a flow channel integrated with the video endoscope. Additionally, the laryngoscope can also includes a suction flow channel having an end located near the distal end of the blade, so as to allow for secretion clearance during intubation.

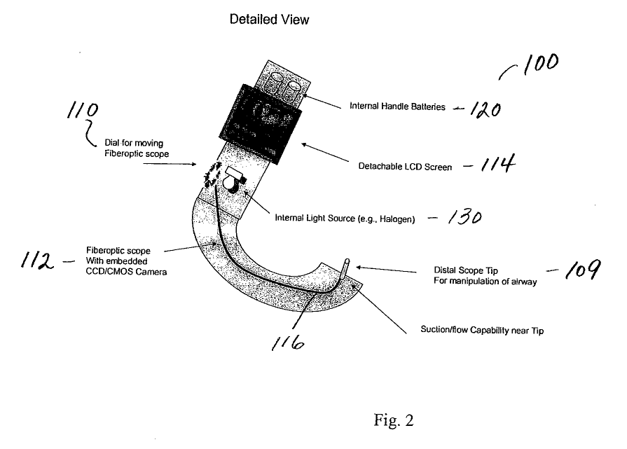

FIG. 2 is a more detailed exemplary diagram for the combined flexible and rigid intubating video laryngoscope of FIG. 1. FIG. 2 shows that the internal portion of the handle 102 includes an illumination source 130. The illumination source 130 can be a halogen or a xenon light source that can be used to provide light to the airway via the optical fibers 116. The internal portion of the handle 102 can also house a source of electrical power 120 (e.g., a battery, or a battery pack) that is sufficiently strong to power the illumination source 130, the display screen 114 and the video endoscope 108.

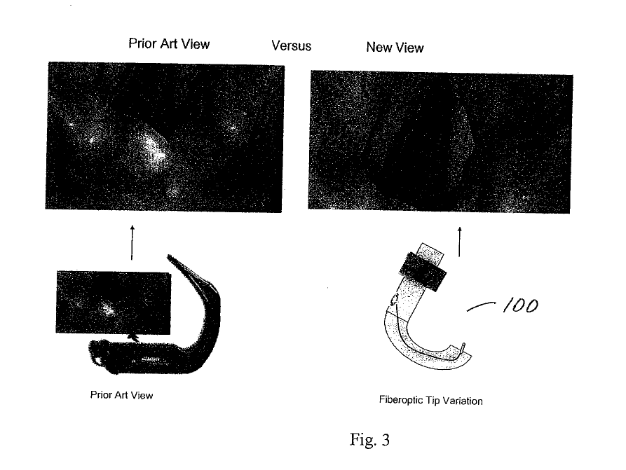

FIG. 3 shows the laryngoscope view using prior art video laryngoscope designs on the left, where the camera is on the tip and looks posteriorly, and the laryngoscope view using the combined flexible and rigid intubating video laryngoscope of FIG. 1, which looks anteriorly. As can be seen from this figure the distal end of the endoscope 109 provides for an adjustable view that can show a more anterior view of the airway. In addition, the attached display screen provides the view of the airway on a display screen that is connected with the handle of the laryngoscope, thus eliminating the need for additional and cumbersome display equipment and associated peripheral devices.

As will be understood by those of skill in the art, the present invention may be embodied in other specific forms without departing from the essential characteristics thereof. For example, any suitable light source, display screen technology or imaging device may be used to enable the combined flexible and rigid intubating video laryngoscope. Furthermore, any apparatus other than a dial and appropriate linkages may be used to vary the field of view of the flexible endoscope at its tip. Accordingly, the foregoing is intended to be illustrative, but not limiting of the scope of the invention, which is set forth in the following claims.

What is claimed is:

1. A combined flexible and rigid intubating video laryngoscope, comprising:
a handle;
a rigid blade, said blade having a proximal end connectible to said handle and a distal end projecting therefrom; and
a flexible video endoscope having a distal end connectible near the distal end of said blade, said distal end of said endoscope being movable relative to said distal end of said blade, so as to enable said endoscope to obtain a variable view including a more anterior anatomical view of a patient's vocal cords during direct laryngoscopy.

2. The laryngoscope of claim 1 further comprising a dial operatively connected with said flexible video endoscope, said dial configured to move said distal end of said endoscope relative to said distal end of said blade.

3. The laryngoscope of claim 1 wherein said flexible video endoscope comprises optic fibers for image transmission and light fibers for airway illumination.

4. The laryngoscope of claim 1 wherein said flexible video endoscope comprises an imaging array.

5. The laryngoscope of claim 4 wherein said imaging array comprises a charge-coupled device imaging array.

6. The laryngoscope of claim 1 further comprising means for flowing a gas to the distal end of said flexible video endoscope.

7. The laryngoscope of claim 6 wherein said means for flowing a gas comprise a flow channel integrated with said video endoscope.

8. The laryngoscope of claim 1 further comprising suction means located near the distal end of said blade, so as to allow for secretion clearance during intubation.

9. The laryngoscope of claim 1 further comprising display means operatively connectible with said video endoscope for displaying the visual field observable by said distal end of said video endoscope.

10. The laryngoscope of claim 9 wherein said display means comprises a display screen detachably connected with said handle.

11. The laryngoscope of claim 9 wherein said display means is positionable to allow the intubator simultaneous viewing of the visual field directly and the visual field indirectly through the display means.

12. The laryngoscope of claim 10 further comprising power supply means placed inside said handle for powering said display screen.

13. The laryngoscope of claim 1 further comprising illumination means placed inside said handle for illuminating the visual field observable by said distal end of said video endoscope.

14. The laryngoscope of claim 13 wherein said illumination means comprises a halogen light source operatively connectible with the proximal end of said video endoscope.

15. The laryngoscope of claim 13 further comprising power supply means placed inside said handle for powering said illumination means and said video endoscope.

16. The laryngoscope of claim 1 further comprising power supply means placed inside said handle for powering said video endoscope.
